SEQUENCE LISTING (4 sequences)

SEQ ID NO: 1 - DNA, 34 bp, Bacteriophage P1
ataacttcgtatagcatacattatacgaagttat

SEQ ID NO: 2 - DNA, 81 bp, Foot-and-mouth disease virus
aaaattgtcgctcctgtcaaacaaactcttaactttgatttactcaaactggctggggatgtagaaagcaatccaggtcca

SEQ ID NO: 3 - DNA, 34 bp, Artificial Sequence
  mutant loxP (lox71) sequence
taccgttcgtatagcatacattatacgaagttat

SEQ ID NO: 4 - DNA, 34 bp, Artificial Sequence
  mutant loxP (lox66) sequence
ataacttcgtatagcatacattatacgaacggta

(19) United States
(12) Patent Application Publication
(10) Pub. No.: US 2011/0003365 A1
(43) Pub. Date: Jan. 6, 2011

(54) METHOD OF PREPARING INDUCED PLURIPOTENT STEM CELLS DEPRIVED OF REPROGRAMMING GENE

(75) Inventors: Shinya YAMANAKA, Kyoto (JP); Keisuke OKITA, Kyoto (JP)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: KYOTO UNIVERSITY, Kyoto-shi (JP)

(21) Appl. No.: 12/790,050

(22) Filed: May 28, 2010

Related U.S. Application Data
(60) Provisional application No. 61/217,284, filed on May 29, 2009.

(51) Int. Cl. C12N 9/90; C12N 5/0735

(52) U.S. Cl. 435/233; 435/325

(57) ABSTRACT

Provided is a method of preparing an induced pluripotent stem cell (iPS cell) deprived of a reprogramming gene, including providing an iPS cell having an expression vector wherein a loxP sequence is placed on each of the 5′ and 3′ sides of the reprogramming gene or a vector component necessary for the replication of the reprogramming gene in the same orientation, and treating the IPS cell with Cre recombinase. Also provided are an iPS cell deprived of a reprogramming gene, as obtained by the method, and a use of the iPS cell as a cell source for producing somatic cells.

### FIELD OF THE INVENTION

The present invention relates to a method of efficiently removing a reprogramming factor from an induced pluripotent stem (hereinafter referred to as iPS) cell transfected with the reprogramming factor, and a method of preparing iPS cells deprived of a reprogramming factor using the foregoing method.

### BACKGROUND OF THE INVENTION

An iPS cell is prepared by transferring a gene known as a reprogramming factor to a somatic cell. Viral vectors such as retroviruses and lentiviruses offer higher gene transfer efficiency than non-viral vectors, and are therefore favorable in that they enable easy preparation of iPS cells.

Meanwhile, retroviruses and lentiviruses become incorporated in the chromosome, posing a problematic with safety in view of the clinical application of the iPS cells prepared using these viral vectors. For this reason, iPS cells prepared using non-viral vectors such as adenoviruses and plasmids without vector incorporation in the chromosome have been reported (1-3). However, these vectors are lower in iPS cell establishment efficiency than retroviruses and lentiviruses. Possibly because of the requirement of persistent high expression of reprogramming factor under iPS cell selection conditions, there are some cases in which a stable expression line having a reprogramming factor incorporated in the chromosome is obtained at a certain frequency even when using a plasmid vector, which is generally recognized as being unlikely to cause the incorporation (2,4).

Hence, attempts have been made to reconcile high establishment efficiency and safety by first establishing an iPS cell using a retrovirus or lentivirus, then removing the extraneous genes from the chromosome. For example, techniques comprising a combination of a lentivirus and the Cre-loxP system have been reported (5, 6). In these reports, however, a complex construct is used wherein a loxP sequence is inserted in the LTR to minimize the risk of activation of an oncogene in the vicinity by an LTR sequence outside the loxP sequence that remains after Cre recombinase treatment, and wherein another promoter such as CMV or EF1α is inserted for transcribing a reprogramming factor; therefore, there is a demand for the development of a vector that can be constructed more easily.

Meanwhile, in the method involving the use of an episomal vector capable of stable self-replication outside the chromosome, the spontaneous clearance of the vector upon discontinuation of drug selection is of low efficiency and takes a long time (3); there is a need for a method of removing the vector in a short time with high efficiency.

### CITED REFERENCES

1. Stadtfeld, M. et al., Science, 322: 945-949 (2008)
2. Okita, K. et al., Science, 322: 949-953 (2008)
3. Yu, J. et al., Science, 324: 797-801 (2009)
4. Kaji, K. et al., Nature, 458: 771-775 (2009)
5. Chang, C. W. et al., Stem Cells, 27: 1042-1049 (2009)
6. Soldner, F. et al., Cell, 136: 964-977 (2009)

### SUMMARY OF THE INVENTION

It is an object of the present invention to provide a method for efficiently depriving an iPS cell incorporating a reprogramming gene of the reprogramming gene, and a method for efficiently preparing a safe iPS cell that is not influenced by the reprogramming factor and has no risk of undergoing an insertion mutation, using the foregoing method.

The present inventors infected mouse hepatocytes or skin fibroblasts with a retrovirus prepared by inserting a reprogramming gene having a loxP sequence placed at each end into a multicloning site in a retrovirus vector, and generated a mouse IFS cell by a conventional method. The iPS cell was then treated with Cre recombinase, and the reprogramming gene was excised from the chromosome. In this case, two LTRs remain on the chromosome, so the chimeric mice generated from the prepared iPS cell were expected to exhibit abnormal phenotypes due to insertion mutations at a certain frequency. Unexpectedly, however, in all the strains examined, the chimeric mice were found to be able to survive long without exhibiting such abnormalities. Thus, the present inventors found for the first time that a safe iPS cell can be prepared efficiently using a viral vector constructed by the very simple procedure of merely inserting a reprogramming gene, flanked by loxP sequences, into a multicloning site of a viral vector.

Furthermore, the present inventors conceptualized that, by applying the Cre-loxP system to an episomal vector, and placing loxP sequences to flank the sequence of a viral protein gene and/or replication origin necessary for the self-replication of the episome, the episome becomes incapable of replicating itself by the action of Cre recombinase, and the vector is diluted and cleared.

Accordingly, the present invention provides the following:

[1] A method of preparing an iPS cell deprived of a reprogramming gene, comprising providing an IPS cell having an expression vector wherein a loxP sequence is placed on each of the 5′ and 3′ sides of the reprogramming gene or a vector component necessary for the replication of the reprogramming gene in the same orientation, and treating the IFS cell with Cre recombinase.
[2] The method according to [1] above, wherein the IPS cell has on the chromosome thereof an expression vector wherein a loxP sequence is placed on each of the 5′ and 3′ sides of the reprogramming gene in the same orientation.
[3] The method according to [2] above, wherein the vector is a retrovirus vector or lentivirus vector.
[4] The method according to [3] above, wherein the loxP sequences are placed inward from the 5′ side LTR and 3′ side LTR, respectively.
[5] The method according to [1] above, wherein the iPS cell has outside the chromosome thereof an expression vector wherein a loxP sequence is placed on each of the 5′ and 3′ sides of the vector component necessary for the replication of the reprogramming gene.
[6] The method according to [5] above, wherein the vector is an episomal vector.
[7] The method according to [6] above, wherein the episomal vector is derived from Epstein-barr virus.
[8] The method according to [7] above, wherein the vector component necessary for the replication of the reprogramming gene is the replication origin oriP.
[9] The method according to [7] above, wherein the vector component necessary for the replication of the reprogramming gene is a gene that encodes EBNA-1.
[10] The method according to [6] above, wherein the episomal vector is derived from SV40.
[11] The method according to [10] above, wherein the vector component necessary for the replication of the reprogramming gene is the replication origin Ori.
[12] The method according to [10] above, wherein the vector component necessary for the replication of the reprogramming gene is a gene that encodes the SV40 large T antigen.
[13] The method according to any one of [1] to [12] above, wherein the loxP sequences are wild type loxP sequences (SEQ ID NO:1).
[14] The method according to any one of [1] to [12] above, wherein the loxP sequences are mutant loxP sequences.
[15] The method according to [14] above, wherein the mutant loxP sequences are combinations of lox71 (SEQ ID NO:3) and lox66 (SEQ ID NO:4).
[16] The method according to any one of [1] to [15] above, wherein the reprogramming gene includes at least one gene selected from among Oct3/4, Sox2, Klf4, c-Myc, Nanog, Lin28 and the SV40 large T antigen.
[17] The method according to [16] above, wherein the reprogramming gene is a polycistronically joined set of 2 or more genes selected from among Oct3/4, Sox2, Klf4, c-Myc, Nanog, Lin28 and the SV40 large T antigen.
[18] The method according to [17] above, wherein the reprogramming gene is a polycistronically joined set of 3 different genes consisting of Oct3/4, Sox2 and Klf4 or 4 different genes consisting of Oct3/4, Sox2, Klf4 and c-Myc.
[19] The method according to [17] or [18] above, wherein the genes are polycistronically joined via the 2A sequence of foot-and-mouth disease virus.
is [20] The method according to any one of [1] to [19] above, wherein the Cre recombinase treatment is carried out by transferring a Cre recombinase expression vector into the iPS cell to allow the enzyme to be produced transiently in the cell.
[21] The method according to [20] above, wherein the Cre recombinase expression vector is a plasmid vector.
[22] An iPS cell deprived of a reprogramming gene, wherein the cell is obtained by the method according to any one of [1] to [4] above and [13] to [21] above.
[23] The iPS cell according to [22] above, wherein the cell has in the intact form the 5′- and 3′-side LTRs harbored by the viral vector.
[24] A use of the iPS cell according to [22] or [23] above in producing a somatic cell.
[25] The iPS cell according to [22] or [23] above as a cell source in producing a somatic cell.

By using a retrovirus or lentivirus in transferring a reprogramming gene into a cell, high gene transfer efficiency is achieved, allowing an iPS cell to be established more efficiently. Furthermore, by removing the reprogramming factor from the chromosome using the Cre-loxP system, a safe iPS cell at reduced risks of insertion mutations and the like can be obtained.

When an episomal vector is used in transferring a reprogramming gene, the reprogramming factor can be removed from the iPS cell in a short time with high efficiency by removing the sequence necessary for the self-replication of the episome using the Cre-loxP system.

### BRIEF DESCRIPTION OF THE DRAWINGS

FIG. 1 is a photographic representation of phase-contrast images and GFP-positive images of iPS cell colonies established by transferring 4 different genes consisting of pMXs-Oct3/4-loxP, pMXs-Sox2-loxP, pMXs-Klf4-loxP, and pMXs-cMyc-loxP (floxed 4 factors), or 3 different genes consisting of pMXs-Oct3/4-loxP, pMXs-Sox2-loxP, and pMXs-Klf4-loxP (floxed 3 factors (-Myc)).

FIG. 2 is a schematic diagram of pMXs-Oct3/4-loxP showing the position of the primers used in genomic PCR analysis. Open arrowheads indicate the position of the LoxP sites. “ψ” indicates packaging signal, “pMX-S1811” indicates forward primer, and “pMX-L3205” indicates reverse primer.

FIG. 3 is a photographic representation of the results of genomic PCR analyses, after Cre treatment, of iPS cells established by transferring 3 different genes consisting of pMXs-Oct3/4-loxP, pMXs-Sox2-loxP, and pMXs-Klf4-loxP.

FIG. 4 is a photographic representation of phase-contrast images and GFP-positive images of 2 different iPS cell clones taken after extraneous gene excision with Cre recombinase.

FIG. 5 is a photographic representation of the results of genomic PCR analyses of iPS cells induced by transferring 3 different genes with mutant loxP vectors (pMXs-Oct3/4-mloxP, pMXs-Sox2-mloxP, and pMXs-Klf4-mloxP) after Cre treatment.

FIG. 6 is a photographic representation of the results of genomic PCR analyses of iPS cells established by 3 different genes consisting of pMXs-Oct3/4-loxP, pMXs-Sox2-loxP, and pMXs-Klf4-loxP (iPS-234D), and iPS cells established by transferring 3 different genes consisting of pMXs-Oct3/4-mloxP, pMXs-Sox2-mloxP, and pMXs-Klf4-mloxP (iPS-283J) after Cre treatment.

FIG. 7 is a schematic diagram of the pMXs plasmid showing the position of the primers used in genomic PCR analysis.

FIG. 8 is a photographic representation of phase-contrast images and GFP-positive images of IPS cell colonies established by transferring 3 different genes with mutant loxP vectors (pMXs-Oct3/4-mloxP, pMXs-Sox2-mloxP, and pMXs-Klf4-mloxP).

FIG. 9 is a photographic representation of colonies of the 2nd subculture shown in FIG. 8.

FIG. 10 is a photographic representation of the results of Southern blot analyses performed using Oct3/4, Sox2 or Klf4 as a probe after the genomic DNA was extracted from each Cre-treated IPS clone and cleaved with BamHI/SphI.

FIG. 11 is a photographic representation of the results of Southern blot analyses performed using the pMXs-5′ probe or pMXs-3′ probe after the genomic DNA of each Cre-treated iPS clone was cleaved with BamHI/EcoRI.

FIG. 12 is a schematic diagram of the pMXs vector integrated in the genome showing the position of the probes used in Southern blot analysis. Restriction enzyme sites are also indicated.

FIG. 13 is a photographic representation of the results of Southern blot analyses performed using pMXs-3′ as a probe after the genomic DNA was extracted from each Cre-treated iPS clone and cleaved with EooRI (283J-3), EcoRI/SphI (283J-4), or EcoRI/SphI (321B-15,-16). The left panel shows the results for iPS cells established by transferring 3 different genes consisting of pMXs-Oct3/4-mloxP, pMXs-Sox2-mloxP, and pMXs-Klf4-mloxP. The right panel shows the results for iPS cells established by transferring 1 gene consisting of pMXs-OKS-mloxP.

FIG. 14 is a schematic diagram of the pMXs vector integrated in the genome showing the position of the probe used in Southern blot analysis.

FIG. 15 is a photographic representation of phase-contrast images and GFP-positive images of iPS cell colonies established by transfection with 2A and mutant loxP vectors. “OKS (mutant loxP)” indicates pMXs-OKS-mloxP, “KOS (mutant loxP)” indicates pMXs-KOS-mloxP, “OK+S (mutant loxP)” indicates pMXs-OK-mloxP and pMXs-Sox2-mloxP, and “O+K+S (mutant loxP)” indicates pMXs-Oct3/4-mloxP, pMXs-Sox2-mloxP, and pMXs-Klf4-mloxP.

FIG. 16 is a photographic representation of colonies of the 2nd subculture shown in FIG. 15.

FIG. 17 is a schematic diagram of pMXs-OKS-mloxP showing the position of the primers used in genomic PCR analysis.

FIG. 18 is a photographic representation of the results of genomic PCR analyses, after Cre treatment, of iPS cells established by transferring pMXs-OKS-mloxP.

FIG. 19 is a photographic representation of the results of a Southern blot analysis performed using pMXs-5′ as a probe after the genomic DNA was extracted from each of the iPS clones 321B-15 and 321B-16 (wherein extraneous gene excision had been confirmed by genomic PCR) and cleaved with BamHI/SphI.

FIG. 20 is a photographic representation of the results of a Southern blot analysis performed using Klf4 as a probe after the genomic DNA was extracted from each the iPS clones 321B-15 and 321B-16 and cleaved with BamHI/SphI.

FIG. 21 is a schematic diagram of the pMXs vector integrated in the genome showing the position of the probes used in Southern blot analysis.

### DETAILED DESCRIPTION OF THE INVENTION

The present invention provides a method of preparing an iPS cell deprived of a reprogramming gene. Here, “a reprogramming gene” means a nucleic acid comprising a nucleotide sequence that encodes a nuclear reprogramming factor. “Deprived of a reprogramming gene” means a state wherein the reprogramming gene does not exist in any of the cell's chromosome and the DNA outside the chromosome.

### (1) First Method of Preparing an iPS Cell

A first method of preparing an iPS cell deprived of a reprogramming gene comprises providing an IPS cell having an expression vector wherein a loxP sequence is placed on each of the 5′ and 3′ sides of the reprogramming gene in the same orientation, and treating the IPS cell with Cre recombinase. This method is applicable to whatever the reprogramming gene has been integrated in the chromosome in the iPS cell or is present outside the chromosome.

### (a) Reprogramming Genes

Examples of preferable of reprogramming genes include, but are not limited to, the following combinations:

(1) Oct3/4, Klf4, c-Myc
(2) Oct3/4, Klf4, c-Myc, Sox2 (here, Sox2 is replaceable with Sox1, Sox3, Sox15, Sox17 or Sox18; Klf4 is replaceable with Klf1, Klf2 or Klf5; c-Myc is replaceable with T58A (active mutant), N-Myc or L-Myc.)
(3) Oct3/4, Klf4, c-Myc, Sox2, Fbx15, Nanog, Eras, ECAT15-2, TclI, β-catenin (active mutant S33Y)
(4) Oct3/4, Klf4, c-Myc, Sox2, TERT, SV40 Large T antigen (hereinafter, SV40LT)
(5) Oct3/4, Klf4, c-Myc, Sox2, TERT, HPV16 E6
(6) Oct3/4, Klf4, c-Myc, Sox2, TERT, HPV16 E7
(7) Oct3/4, Klf4, c-Myc, Sox2, TERT, HPV6 E6, HPV16 E7
(8) Oct3/4, Klf4, c-Myc, Sox2, TERT, Bmil
For details of these combinations, see WO 2007/069666 (however, in the combination (2) above, for replacement of Sox2 with Sox18, and replacement of Klf4 with Klf1 or Klf5, see Nature Biotechnology, 26, 101-106 (2008)); for details of the combination “Oct3/4, Klf4, c-Myc, Sox2”, see also Cell, 126, 663-676 (2006), Cell, 131, 861-872 (2007) and the like: for details of the combination “Oct3/4, Klf2 (or Klf5), c-Myc, Sox2”, see also Nat. Cell Biol., 11, 197-203 (2009); for details of the combination “Oct3/4, Klf4, c-Myc, Sox2, hTERT, SV40LT”, see also Nature, 451, 141-146 (2008)]

### (9) Oct3/4, Klf4, Sox2 (see Nature Biotechnology, 26, 101-106 (2008))

### (10) Oct3/4, Sox2, Nanog, Lin28 (see Science, 318, 1917-1920 (2007))

(11) Oct3/4, Sox2, Nanog, Lin28, hTERT, SV40LT (see Stem Cells, 26, 1998-2005 (2008))
(12) Oct3/4, Klf4, c-Myc, Sox2, Nanog, Lin28 (see Cell Research (2008) 600-603)
(13) Oct3/4, Klf4, c-Myc, Sox2, SV40LT (see also Stem Cells, 26, 1998-2005 (2008))

### (14) Oct3/4, Klf4 (see Nature 454:646-650 (2008), Cell Stem Cell, 2:525-528 (2008))

(15) Oct3/4, c-Myc (see Nature 454:646-650 (2008))

### (16) Oct3/4, Sox2 (see Nature, 451, 141-146 (2008), WO2008/118820)

### (17) Oct3/4, Sox2, Nanog (see WO2008/118820)

### (18) Oct3/4, Sox2, Lin28 (see WO2008/118820)

(19) Oct3/4, Sox2, c-Myc, Esrrb (here, Esrrb is replaceable with Esrrg; see Nat. Cell Biol., 11, 197-203 (2009))
(20) Oct3/4, Sox2, Esrrb (see Nat. Cell Biol., 11, 197-203 (2009))

### (21) Oct3/4, Klf4, L-Myc

### (22) Oct3/4, Nanog

### (23) Oct3/4

(24) Oct3/4, Klf4, c-Myc, Sox2, Nanog, Lin28, SV40LT (see Science, 324: 797-801 (2009))

In (1)-(24) above, in place of Oct3/4, other members of the Oct family, for example, Oct1A, Oct6 and the like, can also be used. In place of Sox2 (or Sox1, Sox3, Sox15, Sox17, Sox18), other members of the Sox family, for example, Sox7 and the like, can also be used. Furthermore, in place of Lin28, other members of the Lin family, for example, Lin28b and the like, can also be used.

Any combination that does not fall in (1) to (24) above but comprises all the constituents of any one of (1) to (24) above and further comprises an optionally chosen other substance can also be included in the scope of “reprogramming genes” in the present invention. Provided that the somatic cell to undergo nuclear reprogramming is endogenously expressing one or more of the constituents of any one of (1) to (24) above at a level sufficient to cause nuclear reprogramming, a combination of only the remaining constituents excluding the one or more constituents can also be included in the scope of “reprogramming genes” in the present invention.

Of these combinations, at least one, preferably 2 or more, more preferably 3 or more, different reprogramming genes selected from among Oct3/4, Sox2, Klf4, c-Myc, Nanog, Lin28 and SV40LT are preferred.

Particularly, if the iPS cells obtained are to be used for therapeutic purposes, the three factors Oct3/4, Sox2 and Klf4 [combination (9) above] are preferably used. If the iPS cells obtained are not to be used for therapeutic purposes (e.g., used as an investigational tool for drug discovery screening and the like), the four factors Oct3/4, Sox2, Klf4 and c-Myc, as well as the five factors Oct3/4, Klf4, c-Myc, Sox2 and Lin28, or the six factors consisting of the five factors and Nanog [combination (12) above] or the seven factors additionally including SV40 Large T [combination (24) above] are preferable.

Furthermore, the above-mentioned combination wherein c-Myc has been changed to L-Myc is also an example of a preferable reprogramming gene.

Information on the mouse and human cDNA sequences of the aforementioned reprogramming genes is available with reference to the NCBI accession numbers mentioned in WO 2007/069666 (in the publication, Nanog is described as ECAT4. Mouse and human cDNA sequence information on Lin28, Lin28b, Esrrb, and Esrrg can be acquired by referring to the following NCBI accession numbers, respectively); those skilled in the art are easily able to isolate these cDNAs.

| Name of gene | Mouse | Human |
|---|---|---|
| Lin28 | NM_145833 | NM_024674 |
| Lin28b | NM_001031772 | NM_001004317 |
| Esrrb | NM_011934 | NM_004452 |
| Esrrg | NM_011935 | NM_001438 |

When 2 or more different reprogramming genes are used, 2 or more, preferably 2 to 4, different genes may be integrated in one expression vector. Alternatively, 2 or more expression vectors incorporating respective different genes may be used. Furthermore, one expression vector incorporating 2 or more different genes and one expression vector incorporating only 1 gene can also be used in combination.

In the context above, when a plurality of reprogramming genes (e.g., 2 or more, preferably 2 to 4 different genes, selected from among Oct3/4, Sox2, Klf4, c-Myc, Nanog, Lin28 and SV40LT, more preferably 3 different genes consisting of Oct3/4, Klf4 and Sox2, or 4 different genes consisting of Oct3/4, Klf4, Sox2 and c-Myc) are integrated in one expression vector, these genes can preferably be integrated into the expression vector via a sequence enabling polycistronic expression. By using a sequence enabling polycistronic expression, it is possible to more efficiently express a plurality of genes integrated in one expression vector. Useful sequences enabling polycistronic expression include, for example, the 2A sequence of foot-and-mouth disease virus (SEQ ID NO:2; PLoS ONE 3, e2532, 2008, Stem Cells 25, 1707, 2007), the IRES sequence (U.S. Pat. No. 4,937,190) and the like, with preference given to the 2A sequence. When a plurality of reprogramming genes are inserted into one expression vector as joined polycistronically, the order of the reprogramming genes is not particularly limited; for example, (i) Oct3/4, Klf4 and Sox2, (ii) Oct3/4, Sox2 and Klf4, (iii) c-Myc, Klf4, Oct3/4 and Sox2, (iv) Oct3/4 and Klf4, (v) Klf4 and Sox2, (vi) Oct3/4 and Sox2, (vii) Sox2 and Klf4, (viii) c-Myc, Lin28 and Nanog, (ix) Oct3/4, Sox2, Nanog and Klf4, (x) Oct3/4, Sox2, SV40 Large T and Klf4, or (xi) c-Myc and Lin28 can be joined together in this order in the orientation from 5′ to 3′.

When a plurality of reprogramming genes are used, at least one thereof can be replaced with a protein encoded thereby as a nuclear reprogramming factor. Transfer of these proteins to somatic cells can be achieved using a method of protein transfer into cells known per se. Such methods include, for example, the method using a protein transfer reagent, the method using a protein transfer domain (PTD)-fusion protein, the microinjection method and the like. Protein transfer reagents are commercially available, including those based on a cationic lipid, such as BioPOTER Protein Delivery Reagent (Gene Therapy Systems), Pro-Ject™ Protein Transfection Reagent (PIERCE) and ProVectin (IMGENEX); those based on a lipid, such as Profect-1 (Targeting Systems); those based on a membrane-permeable peptide, such as Penetrain Peptide (Q biogene) and Chariot Kit (Active Motif), GenomONE (Ishihara Sangyo), which employs the HVJ envelop (inactivated Sendai virus), and the like. The transfer can be achieved per the protocols attached to these reagents, a common procedure being as described below. Nuclear reprogramming substance(s) is (are) diluted in an appropriate solvent (e.g., a buffer solution such as PBS or HEPES), a transfer reagent is added, the mixture is incubated at room temperature for about 5 to 15 minutes to form a complex, this complex is added to cells after exchanging the medium with a serum-free medium, and the cells are incubated at 37° C. for one to several hours. Thereafter, the medium is removed and replaced with a serum-containing medium.

The PTDs developed include those using transcellular domains of proteins such as drosophila-derived AntP, HIV-derived TAT, and HSV-derived VP22. A fusion protein expression vector incorporating a cDNA of a nuclear reprogramming substance and a PTD sequence is prepared to allow the recombinant expression of the fusion protein, and the fusion protein is recovered for use for transfer. This transfer can be achieved as described above, except that no protein transfer reagent is added.

Microinjection, a method of placing a protein solution in a glass needle having a tip diameter of about 1 μm, and injecting the solution into a cell, ensures the transfer of the protein into the cell.

In recent years, methods have been developed for establishing an iPS cell by introducing a reprogramming factor (protein), along with polyarginine or CPPs, into a mouse or human; these techniques can also be used in the present invention (Cell Stem Cell, 4:381-384 (2009), Cell Stem Cell, 4:472-476, doi:10.1016/j.stem.2009.05.005 (2009)).

### (b) Expression Vectors

Reprogramming genes are inserted into an appropriate expression vector harboring a promoter capable of functioning in the somatic cell to be transfected therewith. Useful expression vectors include, for example, viral vectors such as retroviruses (e.g., pMX), lentiviruses (e.g., pKP114), adenoviruses, adeno-associated viruses and herpesviruses; episomal vectors capable of self-replication, derived from EBV, SV40, Sendai virus and the like; and plasmids for the expression in animal cells (e.g., pA1-11, pXT1, pRc/CMV, pRc/RSV, pcDNAI/Neo) and the like. If integration into the chromosome is intended initially, it is preferable to use a retrovirus or lentivirus because of the high iPS cell establishment efficiency. However, the present invention can also be used preferably for removing a reprogramming gene from an iPS cell wherein the reprogramming gene has been integrated in the chromosome contrary to expectations when using an adenovirus or plasmid vector, and also for removing a reprogramming gene from the episomal vector in a short time with high efficiency.

Examples of promoters used in the expression vectors include the SRα promoter, the SV40 early promoter, the retrovirus LTR, the CMV (cytomegalovirus) promoter, the RSV (Rous sarcoma virus) promoter, the HSV-TK (herpes simplex virus thymidine kinase) promoter, the EF1α promoter, the metallothionein promoter, the heat shock promoter and the like. An enhancer of the IE gene of human CMV may be used along with a promoter. For example, the CAG promoter (comprising a cytomegalovirus enhancer, the chicken β-actin promoter and β-globin gene poly-A signal site) can be used.

In addition to a promoter, the expression vector may contain as desired an enhancer, a polyA addition signal, a selection marker gene, a replication origin, a gene that encodes a protein that binds to a replication origin to control the replication, and the like. Examples of selection marker genes include the dihydrofolate reductase gene and the neomycin resistance gene.

(c) loxP Sequences

The loxP sequences useful in the present invention include, in addition to the bacteriophage P1-derived wild type loxP sequence (SEQ ID NO:1), optionally chosen mutant loxP sequences capable of deleting the floxed sequence (sequence flanked by the loxP sequences) by recombination by Cre recombinase when placed in the same orientation at positions flanking the reprogramming gene (in the second method described below, a vector component necessary for the replication of the reprogramming gene). Examples of such mutant loxP sequences include lox77 (SEQ ID NO:3), mutated in 5′ repeat, lox66 (SEQ ID NO:4), mutated in 3′ repeat, and lox2272 and lox511, mutated in spacer portion. Although the two loxP sequences placed on the 5′ and 3′ sides of the reprogramming factor may be identical or not, the two mutant loxP sequences mutated in spacer portion must be identical (e.g., a pair of lox2272 sequences, a pair of lox511 sequences). Preference is given to a combination of a mutant loxP sequence mutated in 5′ repeat (e.g., lox71) and a mutant loxP sequence mutated in 3′ repeat (e.g., lox66). In this case, the loxP sequences remaining on the chromosome have double mutations in the repeats on the 5′ side and 3′ side as a result of recombination by Cre recombinase, and are therefore unlikely to be recognized by Cre recombinase, thus reducing the risk of causing a deletion mutation in the chromosome due to unwanted recombination. When the mutant loxP sequences lox71 and lox66 are used in combination, each may be placed on any of the 5′ and 3′ sides of the reprogramming gene, but it is necessary that the mutant loxP sequences be inserted in an orientation such that the mutated sites would be located at the outer ends of the respective loxP sequences.

The two loxP sequences can be inserted into optionally chosen positions in the expression vector, provided that they flank the reprogramming gene (or the cassette of a plurality of reprogramming genes joined together), and that the loxP sequences also become integrated in the chromosome if the reprogramming gene is integrated in the chromosome. In the case of a retrovirus or lentivirus, one loxP sequence can be placed at an optionally chosen position between the 5′ end of the reprogramming gene and the inside of the LTR outside the reprogramming gene, and the other loxP sequence can be placed at an optionally chosen position between the 3′ end of the reprogramming gene and the inside of the LTR outside the reprogramming gene. To minimize the viral vector sequence that remains on the chromosome after recombination by the Cre recombinase treatment, it is preferable to place the loxP sequences in LTRs. When the loxP sequences are to be placed in LTRs, a loxP sequence would be located in each of the two LTRs appearing at the respective ends of the vector integrated in the chromosome as a result of gene duplication during pro-virus replication by previously placing a loxP sequence in either the 5′ LTR or 3′ LTR. In this case, the LTRs incorporating one loxP have their promoter/enhancer activity reduced or lost, so it is desirable that another promoter (e.g., SRα promoter, CMV IE promoter, CAG promoter, EF1α promoter and the like) be placed at a position that is in the LTRs, and that allows the transcription of the reprogramming gene to be controlled. Because an enhancer-promoter sequence in the LTR U3 region possibly upregulates the host gene in the vicinity thereof by an insertion mutation, it is more preferable to avoid the control of the expression of endogenous genes by LTRs outside the loxP sequences remaining in the genome without excision, using a 3′-self-inactivated (SIN) LTR prepared by deleting the sequence or replacing the sequence with a polyadenylating sequence such as SV40. A specific means using SIN LTR is described in Stem Cells, 27: 1042-1049 (2009).

In another preferred embodiment of the present invention, however, the two loxP sequences are placed inward from the LTRs, more preferably at positions adjacent to the 5′ and 3′ ends of the reprogramming gene. The present invention is based, at least partially, on the discovery that after the reprogramming gene is excised by Cre recombinase treatment, even if the 5′ and/or 3′ LTR of the retrovirus or lentivirus remains in its intact form on the chromosome, the risk of causing abnormalities due to insertion mutation is extremely low. This fact shows that a safe iPS cell can be prepared at high probability merely by using a reprogramming gene with a loxP sequence added to each end of the reprogramming gene, inserted into a multicloning site in a publicly known retrovirus or lentivirus expression vector, even without constructing an expression vector by painstaking genetic operations such as inserting loxP sequences in LTRs, and further placing another promoter that controls the transcription of the reprogramming gene.

### (d) Sources of Somatic Cells

Any cells, but other than germ cells, of mammalian origin (e.g., mice, humans) can be somatic cells used as starting material for the production of iPS cells in the present invention. Examples include keratinizing epithelial cells (e.g., keratinized epidermal cells), mucosal epithelial cells (e.g., epithelial cells of the superficial layer of tongue), exocrine gland epithelial cells (e.g., mammary gland cells), hormone-secreting cells (e.g., adrenomedullary cells), cells for metabolism or storage (e.g., liver cells), intimal epithelial cells constituting interfaces (e.g., type I alveolar cells), intimal epithelial cells of the obturator canal (e.g., vascular endothelial cells), cells having cilia with transporting capability (e.g., airway epithelial cells), cells for extracellular matrix secretion (e.g., fibroblasts), constrictive cells (e.g., smooth muscle cells), cells of the blood and the immune system (e.g., T lymphocytes), sense-related cells (e.g., rod cells), autonomic nervous system neurons (e.g., cholinergic neurons), sustentacular cells of sensory organs and peripheral neurons (e.g., satellite cells), nerve cells and glia cells of the central nervous system (e.g., astroglia cells), pigment cells (e.g., retinal pigment epithelial cells), progenitor cells thereof (tissue progenitor cells) and the like. There is no limitation on the degree of cell differentiation; even undifferentiated progenitor cells (including somatic stem cells) and finally differentiated mature cells can be used alike as sources of somatic cells in the present invention. Examples of undifferentiated progenitor cells include tissue stem cells (somatic stem cells) such as nerve stem cells, hematopoietic stem cells, mesenchymal stem cells, and dental pulp stem cells.

The choice of mammal as a source of somatic cells is not particularly limited; however, when the iPS cells obtained are to be used for regenerative medicine in humans, it is particularly preferable, from the viewpoint of prevention of graft rejection, that somatic cells are patient's own cells or collected from another person having the same HLA type as that of the patient. When the iPS cells obtained are not to be administered (transplanted) to a human, but used as, for example, a source of cells for screening for evaluating a patient's drug susceptibility or adverse reactions, it is likewise necessary to collect the somatic cells from the patient or another person with the same genetic polymorphism correlating with the drug susceptibility or adverse reactions.

(e) Method of Introducing a Reprogramming Gene into Somatic Cell

An expression vector harboring a reprogramming gene can be introduced into a cell by a technique known per se according to the choice of the vector. In the case of a viral vector, for example, a plasmid containing the nucleic acid is introduced into an appropriate packaging cell (e.g., Plat-E cells) or a complementary cell line (e.g., 293 cells), the viral vector produced in the culture supernatant is recovered, and the vector is infected to the cell by a method suitable for the viral vector. For example, specific means using a retroviral vector as a vector are disclosed in WO2007/69666, Cell, 126, 663-676 (2006) and Cell, 131, 861-872 (2007). Specific means using a lentivirus vector as a vector is disclosed in Science, 318, 1917-1920 (2007). Specific means using an adenoviral vector is described in Science, 322, 945-949 (2008).

Meanwhile, in the case of a non-viral vector such as a plasmid vector or an episomal vector having a virus-derived self-replication mechanism, the vector can be transferred into a cell using the lipofection method, liposome method, electroporation method, calcium phosphate co-precipitation method, DEAE dextran method, microinjection method, gene gun method and the like. Specific means using a plasmid as a vector are described in, for example, Science, 322, 949-953 (2008) and the like. Specific means using an episomal vector as a vector are described in, for example, Science, 324: 797-801 (2009) and the like.

When a plasmid vector, adenovirus vector or the like is used, the transfecting operation can be performed once or more optionally chosen times (e.g., once or more to 10 times or less, or once or more to 5 times or less and the like). When 2 kinds or more of expression vectors are transferred to a somatic cell, it is preferable that all these expression vectors be introduced into the somatic cell at one time. In this case as well, the transfecting operation can be performed once or more optionally chosen times (e.g., once or more to 10 times or less, or once or more to 5 times or less and the like), preferably twice or more (e.g., 3 times or 4 times).

### (f) iPS Cell Establishment Efficiency Improvers

In recent years, various substances that improve the efficiency of establishment of iPS cells, which has traditionally been low, have been proposed one after another. When brought into contact with a somatic cell together with the aforementioned reprogramming genes, these establishment efficiency improvers are expected to further raise the efficiency of establishment of iPS cells.

Examples of iPS cell establishment efficiency improvers include, but are not limited to, histone deacetylase (HDAC) inhibitors [e.g., valproic acid (VPA) (Nat. Biotechnol., 26(7): 795-797 (2008)], low-molecular inhibitors such as trichostatin A, sodium butyrate, MC 1293, and M344, nucleic acid-based expression inhibitors such as siRNAs and shRNAs against HDAC (e.g., HDAC1 siRNA Smartpool® (Millipore), HuSH 29mer shRNA Constructs against HDAC1 (OriGene) and the like), and the like], DNA methyltransferase inhibitors (e.g., 5′-azacytidine) [Nat. Biotechnol., 26(7): 795-797 (2008)], G9a histone methyltransferase inhibitors [e.g., low-molecular inhibitors such as BIX-01294 (Cell Stem Cell, 2: 525-528 (2008)], nucleic acid-based expression inhibitors such as siRNAs and shRNAs against G9a [e.g., G9a siRNA (human) (Santa Cruz Biotechnology) and the like) and the like], L-channel calcium agonists (e.g., Bayk8644) [Cell Stem Cell, 3, 568-574 (2008)], p53 inhibitors [e.g., siRNA and shRNA against p53 (Cell Stem Cell, 3, 475-479 (2008)), UTF1 [Cell Stem Cell, 3, 475-479 (2008)], Wnt Signaling (e.g., soluble Wnt3a) [Cell Stem Cell, 3, 132-135 (2008)], 2i/LIF [2i is an inhibitor of mitogen-activated protein kinase signaling and glycogen synthase kinase-3, PloS Biology, 6(10), 2237-2247 (2008)] and the like. As mentioned above, the nucleic acid-based expression inhibitors may be in the form of expression vectors harboring a DNA that encodes an siRNA or shRNA.

Among the constituents of the aforementioned reprogramming genes, SV40LT and the like, for example, can also be included in the scope of iPS cell establishment efficiency improvers because they are deemed not essential, but auxiliary, factors for somatic cell nuclear reprogramming. In the situation of the mechanisms for nuclear programming remaining unclear, the auxiliary factors, which are not essential for nuclear reprogramming, may be conveniently considered as nuclear reprogramming factors or iPS cell establishment efficiency improvers. Hence, because the somatic cell nuclear reprogramming process is understood as an overall event resulting from contact of nuclear reprogramming factor(s) and IPS cell establishment efficiency improver(s) with a somatic cell, it seems unnecessary for those skilled in the art to always distinguish between the nuclear reprogramming substance and the IPS cell establishment efficiency improver.

Contact of an iPS cell establishment efficiency improver with a somatic cell can be achieved as described above for each of the reprogramming genes and nuclear reprogramming factors (proteins) substituting the genes, when (a) the improver is a proteinous factor or (b) the improver is a nucleic acid that encodes the proteinous factor. In addition, when the establishment efficiency improver is (c) a low-molecular compound, it can be achieved by dissolving the substance at an appropriate concentration in an aqueous or non-aqueous solvent, adding the solution to a medium suitable for cultivation of somatic cells isolated from human or mouse [e.g., minimal essential medium (MEM) comprising about 5 to 20% fetal calf serum, Dulbecco's modified Eagle medium (DMEM), RPMI1640 medium, 199 medium, F12 medium and the like] such that the concentration of the substance falls in a range that is sufficient to improve the iPS cell establishment efficiency and does not cause cytotoxicity, and culturing the cells for a given period. The concentration of the substance for improving the iPS cell establishment efficiency varies depending on the kind of the substance to be used, and is chosen as appropriate from the range of about 0.1 nM to about 100 nM. Duration of contact is not particularly limited, as far as it is sufficient to cause nuclear reprogramming of the cells; usually, the substance may be co-present in the medium until a positive colony emerges.

An iPS cell establishment efficiency improver may be brought into contact with a somatic cell simultaneously with a reprogramming gene, or either one may be contacted in advance, as far as the efficiency of establishment of iPS cells from the somatic cell is significantly improved, compared with the absence of the improver. In an embodiment, for example, when the iPS cell establishment efficiency improver is a low molecular weight compound, the iPS cell establishment efficiency improver can be added to the medium after the cell is cultured for a given length of time after the gene transfer treatment, because the reprogramming gene involves a given length of time lag from the gene transfer treatment to the mass-expression of the proteinous factor, whereas the establishment efficiency improver is capable of rapidly acting on the cell. In another embodiment, when a reprogramming gene and an iPS cell establishment efficiency improver are both used in the form of a viral or non-viral vector, for example, both may be simultaneously introduced into the cell.

(g) Cultivation of Somatic Cells Before and after Transfection and Selection of iPS Cells

Somatic cells separated from a mouse or human can be pre-cultured using a medium known per se suitable for the cultivation thereof, depending on the kind of the cells, prior to applying to nuclear reprogramming step. Examples of such media include, but are not limited to, a minimal essential medium (MEM) containing about 5 to 20% fetal calf serum, Dulbecco's modified Eagle medium (DMEM), RPMI1640 medium, 199 medium, F12 medium and the like. When using, for example, a transfection reagent such as a cationic liposome in contacting the cell with reprogramming gene(s) and iPS cell establishment efficiency improver(s), it is sometimes preferable that the medium be previously replaced with a serum-free medium to prevent a reduction in the transfer efficiency. After the reprogramming gene(s) (and iPS cell establishment efficiency improver(s)) is (are) brought into contact with the cell, the cell can be cultured under conditions suitable for the cultivation of, for example, ES cells. In the case of mouse cells, the cultivation is carried out with the addition of Leukemia Inhibitory Factor (LIF) as a differentiation suppressor to an ordinary medium. Meanwhile, in the case of human cells, it is desirable that basic fibroblast growth factor (bFGF) and/or stem cell factor (SCF) be added in place of LIF. Usually, the cells are cultured in the co-presence of mouse embryo-derived fibroblasts (MEFs) treated with radiation or an antibiotic to terminate the cell division thereof, as feeder cells. Usually, STO cells and the like are commonly used as MEFs but for inducing iPS cells, SNL cells [McMahon, A. P. & Bradley, A. Cell 62, 1073-1085 (1990)] and the like are commonly used. Co-culture with feeder cells may be started before contact of the nuclear reprogramming substance, at the time of the contact, or after the contact (e.g., 1-10 days later).

A candidate colony of iPS cells can be selected by a method with drug resistance and reporter activity as indicators, and also by a method based on visual examination of morphology. As an example of the former, a colony positive for drug resistance and/or reporter activity is selected using a recombinant somatic cell wherein a drug resistance gene and/or a reporter gene is targeted to the locus of a gene highly expressed specifically in pluripotent cells (e.g., Fbx15, Nanog, Oct3/4 and the like, preferably Nanog or Oct3/4). Examples of such recombinant somatic cells include MEFs from a mouse having the βgeo (which encodes a fusion protein of β-galactosidase and neomycin phosphotransferase) gene knocked-in to the Fbx15 locus [Takahashi & Yamanaka, Cell, 126, 663-676 (2006)], MEFs from a transgenic mouse having the green fluorescent protein (GFP) gene and the puromycin resistance gene integrated in the Nanog locus [Okita et al., Nature, 448, 313-317 (2007)] and the like. Meanwhile, examples of the latter method based on visual examination of morphology include the method described by Takahashi et al. in Cell, 131, 861-872 (2007). Although the method using reporter cells is convenient and efficient, it is desirable from the viewpoint of safety that colonies be selected by visual examination when iPS cells are prepared for the purpose of human treatment. When the three factors Oct3/4, Klf4 and Sox2 are used as reprogramming genes, the number of clones established decreases but the resulting colonies are mostly of iPS cells of high quality comparable to ES cells, so that iPS cells can efficiently be established even without using reporter cells.

The identity of the cells of a selected colony as iPS cells can be confirmed by positive responses to a Nanog (or Oct3/4) reporter (puromycin resistance, GFP positivity and the like) and by the formation of a visible ES cell-like colony, as described above. However, to ensure higher accuracy, it is possible to perform tests such as analyzing the expression of various ES-cell-specific genes and transplanting the cells selected to a mouse and confirming the formation of teratomas. All these test methods are obvious.

### (h) Cre Recombinase Treatment

Useful methods of treating an iPS cell obtained as described above with Cre recombinase include (a) a method wherein a Cre recombinase expression vector is transferred to the iPS cell to allow the enzyme to be produced transiently in the cell, or (b) a method wherein Cre recombinase is brought into contact with the IPS cell (preferably, using the aforementioned protein transfer reagent and the like) to supply the Cre recombinase into the cell, with preference given to the method (a). Examples of vectors capable of transiently expressing Cre recombinase include plasmid vectors, adenoviral vectors and the like, with preference given to plasmid vectors. Preferable plasmid vectors are exemplified by those used in transferring a reprogramming gene.

### (i) Confirmation of Removal of Reprogramming Gene

Whether or not the reprogramming gene has been removed from the iPS cell by the Cre recombinase treatment can be confirmed by performing a Southern blot analysis or PCR analysis using a nucleic acid comprising a nucleotide sequence in the reprogramming gene and/or in the vicinity of loxP sequence as a probe or primer, with chromosome DNA and/or episome fraction isolated from the iPS cell as a template, to determine the presence or absence of a band or the length of the band detected. Specific procedures are described in Examples below.

### (2) Second Method of Preparing an iPS Cell

A second method of preparing an iPS cell deprived of a reprogramming gene comprises providing an iPS cell having an expression vector wherein a loxP sequence is placed on each of the 5′ and 3′ sides of a vector component necessary for the replication of the reprogramming gene in the same orientation, and treating the iPS cell with Cre recombinase. This method can be used when the reprogramming gene is present as an episome in the iPS cell.

In the second method, the choice of (a) reprogramming gene, (c) kind of loxP sequences, (d) somatic cell source and (f) IPS cell establishment efficiency improvers used to prepare the IPS cell, (g) cultivation of somatic cells before and after transduction and selection of iPS cells, and (h) Cre recombinase treatment are the same as those used in the first method described above. The (b) expression vector used to prepare an iPS cell is exemplified by episomal vectors, for example, a vector comprising as a vector component a sequence derived from EBV, SV40 and the like necessary for self-replication. The vector component necessary for self-replication is specifically exemplified by a replication origin and a gene sequence that encodes a protein that binds to a replication origin to control the replication; examples include the replication origin oriP and the EBNA-1 gene for EBV, and the replication origin on and the SV40 large T antigen gene for SV40.

The episomal expression vector comprises a promoter that controls the transcription of the reprogramming gene. Useful promoters include those mentioned as examples of expression vectors used in the first method described above. The episomal expression vector, as with the expression vector used in the aforementioned first method, may further contain as desired an enhancer, a polyA addition signal, a selection marker gene, a replication origin, a gene that encodes a protein that binds to a replication origin to control the replication, and the like. Examples of useful selection marker genes include the dihydrofolate reductase gene, the neomycin resistance gene and the like.

In the second method, each of the two loxP sequences is placed on the 5′ and 3′ sides of a vector component necessary for the replication of the reprogramming gene (i.e., a replication origin, or a gene sequence that encodes a protein that binds to a replication origin to control the replication) in the same orientation. The vector component flanked by the loxP sequences may be either a replication origin or a gene sequence that encodes a protein that binds to a replication origin to control the replication, or both.

In the second method, the reprogramming gene allows the vector to be introduced into the cell using, for example, the lipofection method, liposome method, electroporation method, calcium phosphate co-precipitation method, DEAE dextran method, microinjection method, gene gun method and the like. Specifically, for example, methods described in Science, 324: 797-801 (2009) and elsewhere can be used.

Whether or not the vector component necessary for the replication of the reprogramming gene has been removed from the iPS cell by the Cre recombinase treatment can be confirmed by performing a Southern blot analysis or PCR analysis using a nucleic acid comprising a nucleotide sequence in the vector component and/or in the vicinity of loxP sequence as a probe or primer, with the episome fraction isolated from the IFS cell as a template, and determining the presence or absence of a band or the length of the band detected. The episome fraction can be prepared by a method obvious in the art; for example, methods described in Science, 324: 797-801 (2009) and elsewhere can be used.

An IPS cell having a genome structure wherein after a reprogramming gene is integrated in the chromosome using a retrovirus or lentivirus, only the reprogramming gene is removed from the chromosome by a deletion mutation using the Cre-loxP system, while allowing the LTRs at both ends to remain in the intact form, is a novel cell distinct from conventionally known IPS cells.

The IFS cells thus established can be used for various purposes. For example, by utilizing a reported method of differentiation induction for ES cells, differentiation of the iPS cells into various cells (e.g., myocardial cells, blood cells, nerve cells, vascular endothelial cells, insulin-secreting cells and the like) can be induced. Therefore, inducing iPS cells using somatic cells collected from a patient would enable stem cell therapy based on autologous transplantation, wherein the iPS cells are differentiated into desired cells (cells of an affected organ of the patient, cells that have a therapeutic effect on disease, and the like), and the differentiated cells are transplanted to the patient. Somatic cells collected not from a patient, but from another person with the same HLA type as that of the patient, may be used to induce iPS cells, which are differentiated into desired cells for use in transplantation to the patient. Furthermore, because functional cells (e.g., liver cells) differentiated from iPS cells are thought to better reflect the actual state of the functional cells in vivo than do corresponding existing cell lines, they can also be suitably used for in vitro screening for the effectiveness and toxicity of pharmaceutical candidate compounds and the like.

The present invention is hereinafter described in further detail by means of the following examples, to which, however, the invention is never limited.

### EXAMPLES

### Example 1

### Preparation of Reprogramming Gene Expression Retrovirus Vectors Harboring loxP Sequences

Retrovirus vectors used for reprogramming were prepared using pMXs (obtained from Professor Toshio Kitamura at the University of Tokyo; Exp. Hematol. 31; 1007-1014, 2003). Constructs were prepared by flanking each of the coding regions of mouse-derived Oct3/4, Sox2, Klf4 and c-Myc with loxP sequences (5′-ataacttcgtatagcatacattatacgaagttat-3′, SEQ ID NO:1). Each of the constructs was inserted into a multicloning site of the vector, whereby retrovirus vectors that express the respective reprogramming genes were prepared (pMXs-Oct3/4-loxP, pMXs-Sox2-loxP, pMXs-Klf4-loxP, pMXs-cMyc-loxP). Likewise, constructs were prepared by flanking each of the coding regions of mouse-derived Oct3/4, Sox2 and Klf4 with mutant loxP sequences, and each of the constructs was inserted, whereby retrovirus vectors that express the respective reprogramming genes were prepared (pMXs-Oct3/4-mloxP, pMXs-Sox2-mloxP, pMXs-Klf4-mloxP).

Furthermore, constructs were prepared by joining the translated regions of the foregoing 3 or 2 different genes flanking the 2A sequence of foot-and-mouth disease virus (aaaattgtcg ctcctgtcaa acaaactctt aactttgatt tactcaaact ggctggggat gtagaaagca atccaggtcc a, SEQ ID NO:2), and each end of the construct was flanked with mutant loxP sequences and inserted, whereby vectors were prepared (pMXs-OKS-mloxP, pMXs-KOS-mloxP, pMXs-OK-mloxP).

The mutant loxP sequences used were lox71 (5′-taccgttcgtatagcatacattatacgaagttat-3′, SEQ ID NO:3) and lox66 (5′-ataacttcgtatagcatacattatacgaacggta-3′, SEQ ID NO:4) (Plant J. 7; 649-659, 1995). For the constructs prepared by joining the components with the 2A sequence, lox66 was used on the 5′ side, and lox71 on the 3′ side (both inserted in the reverse orientation). For the other constructs (constructs of each gene alone), lox71 was used on the 5′ side, and lox66 on the 3′ side. These mutant loxP sequences become unlikely to be recognized by Cre after recombination by Cre recombinase, and are therefore thought to be unlikely to undergo unwanted recombination.

### Example 2

### Induction of IPS Cells from Mouse Hepatocytes (Exp. Nos. 234 and 296)

Hepatocytes from a Nanog reporter mouse (Okita K. et al., Nature 448, 313-317 (2007)) were used in the experiments. This mouse has a Nanog reporter prepared by integrating green fluorescent protein (EGFP) and the puromycin resistance gene into the Nanog gene locus of a BAC (bacterial artificial chromosome) purchased from BACPAC Resources. The mouse Nanog gene is expressed specifically in pluripotent cells such as ES cells and early embryos, and mouse iPS cells positive for this reporter have been shown to possess a differentiating potential nearly equivalent to that of ES cells.

1.25×10⁵ hepatocytes from the Nanog reporter mouse were sown onto each well of a 6-well culture plate containing previously sown feeder cells (puromycin- and hygromycin-resistant MSTO cells, hereinafter simply referred to as MSTO cells). The cells were cultured in DMEM/10% FCS culture broth at 37° C. and in the presence of 5% CO₂. Two days later, the cells were transfected with the following retrovirus vectors prepared in Example 1. This transfection (viral infection) was performed as described in Nature 448, 313-317 (2007).

(1) 4 different genes consisting of pMXs-Oct3/4-loxP, pMXs-Sox2-loxP, pMXs-Klf4-loxP, and pMXs-cMyc-loxP
(2) 3 different genes consisting of pMXs-Oct3/4-loxP, pMXs-Sox2-loxP, and pMXs-Klf4-loxP
(3) 3 different genes consisting of pMXs-Oct3/4-mloxP, pMXs-Sox2-mloxP, and pMXs-Klf4-mloxP

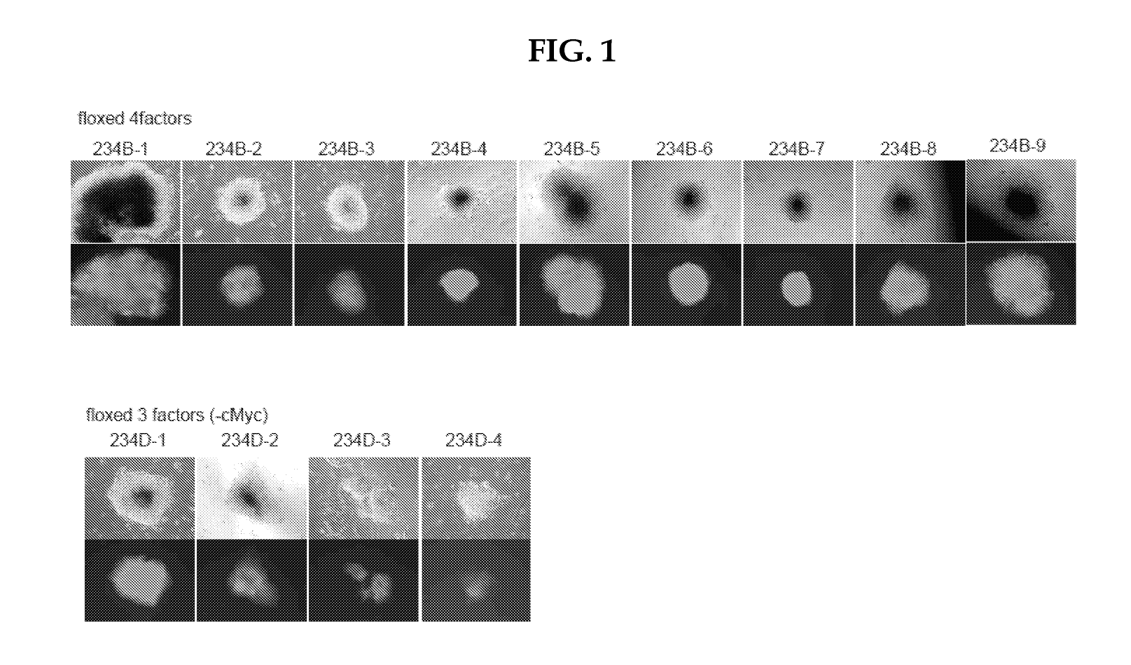

On day 18 of viral infection, selection with puromycin (1.5 μg/mL) was started. On day 24, colonies were picked up. The results of transfer of the 4 genes (1) above (floxed 4 factors) and the results of transfer of the 3 genes (2) above (floxed 3 factors) are shown in FIG. 1. All colonies obtained exhibited a typical ES-cell-like morphology and tested positive for GFP, demonstrating the establishment of iPS cells. In the case of transfer of the 3 genes (3) above as well, establishment of similar IPS cells was confirmed.

Subsequently, 30 μg of pCAG-Cre-Hyg (a vector expressing Cre recombinase under the control of CAG promoter, and expressing hygromycin under the control of PGK promoter) was electrically introduced into 1×10⁷ iPS cells of the 8th subculture (Bio-Rad Company: GenePulser Xcell). One-third of the cells were sown onto a 100 mm dish containing previously sown MSTO cells. On day 2 to day 4 after the transfection, the cells were treated with hygromycin, whereby Cre expressing cells were selected. On day 12, iPS colonies were picked up; to confirm extraneous gene excision by Cre, genomic PCR analyses were performed.

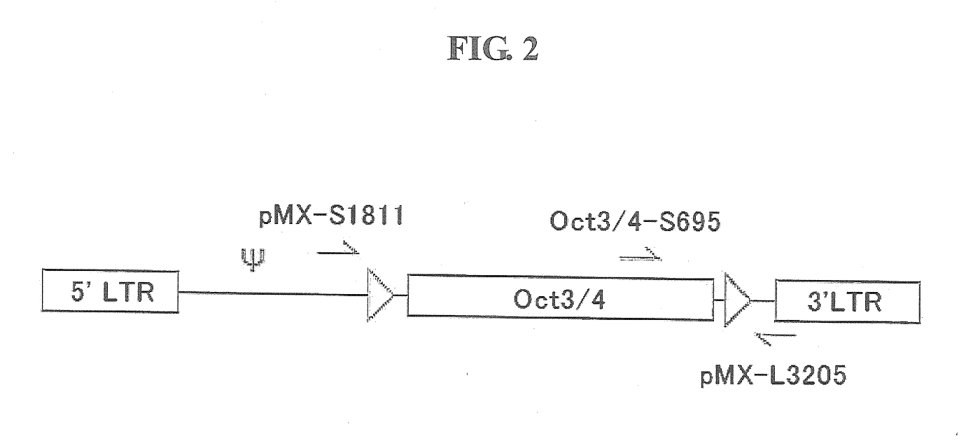

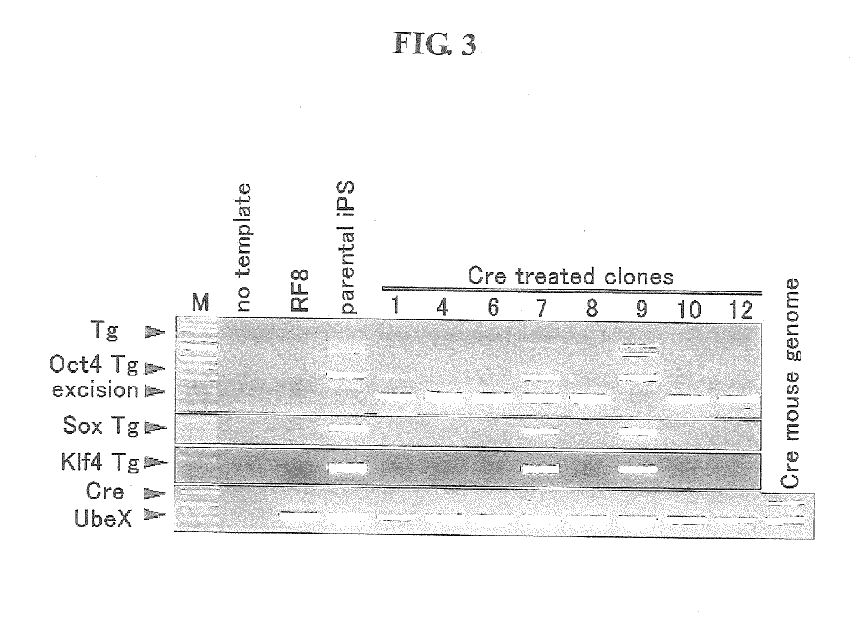

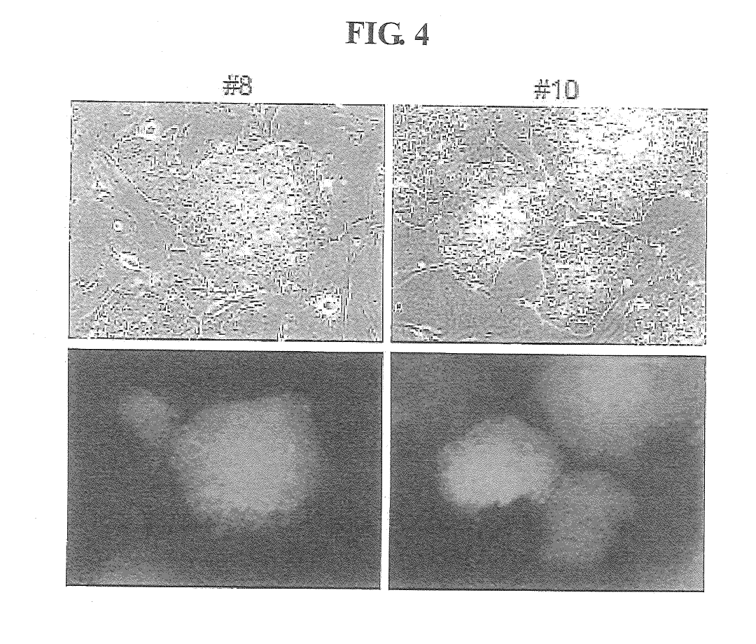

A schematic diagram of the plasmid pMXs-Oct3/4-loxP showing the position of the primers used in the genomic PCR analysis is given in FIG. 2. The results of the PCR analysis of iPS cells transfected with the 3 genes (2) above, and treated with Cre, are shown in FIG. 3. In 5 of the 8 different Cre-treated iPS clones, extraneous gene excision by the Cre-loxP reaction was confirmed (clones that produced bands only at the “excision” position in the figure: 4, 6, 8, 10, 12). The iPS clones after extraneous gene excision were found to maintain a morphology for iPS cells and GFP positivity even after being cultured thereafter (FIG. 4).

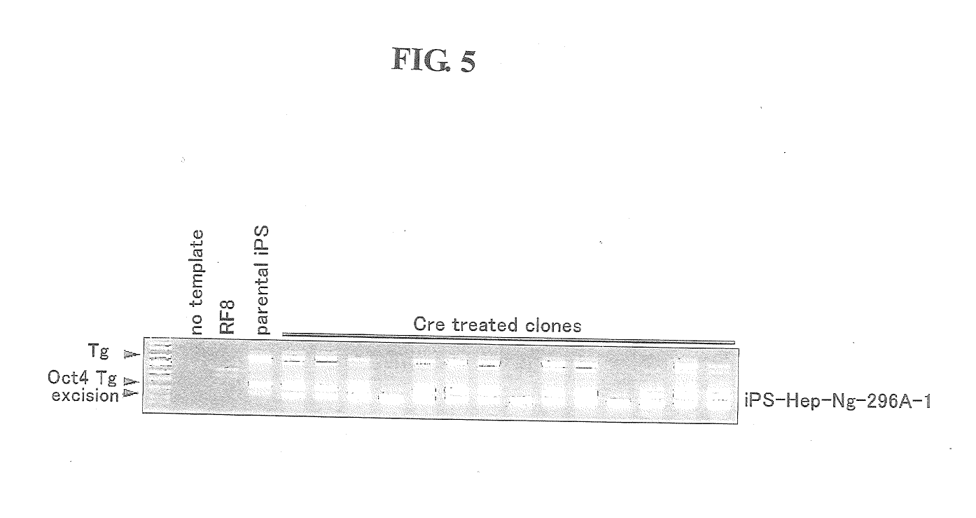

The results of the genomic PCR analysis of the iPS cell induced by the transferring 3 genes (3) above with mutant loxP vectors after Cre treatment are shown in FIG. 5. For 3 different clones, extraneous gene excision by the Cre-loxP reaction was confirmed (clones that produced a band only at the “excision” position in the figure).

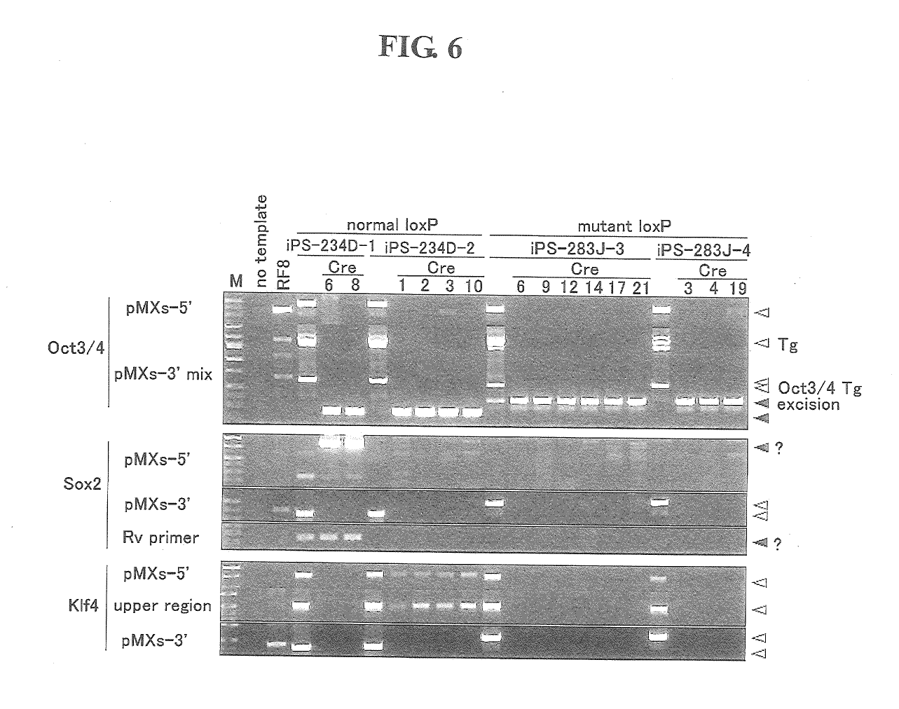

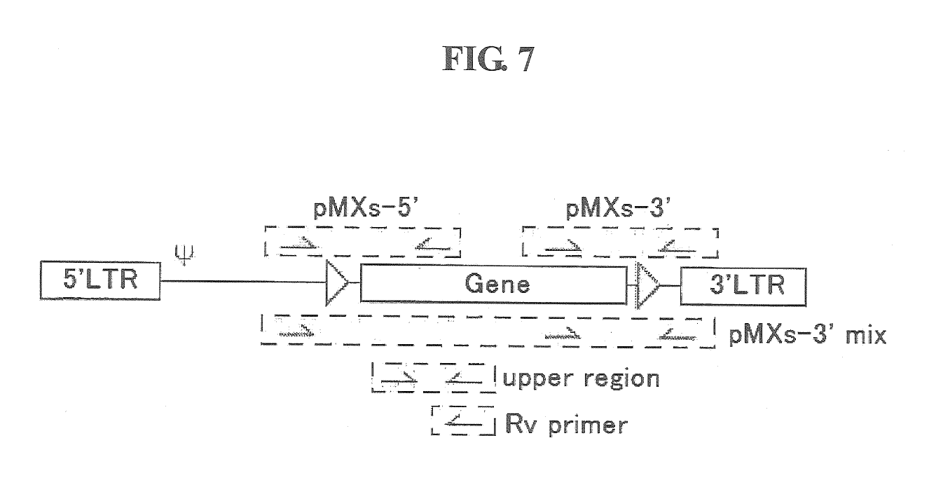

The results of a more exact confirmation of extraneous gene excision using other primers are shown in FIG. 6 (left). A schematic diagram of the pMXs plasmid showing the position of the primer used in the genomic PCR analysis is given in FIG. 7.

In iPS-234D-1, obtained in an experiment of (2) above, it was estimated from the band pattern that the retrovirus had been integrated in an incomplete form; however, in the 2 clones examined, extraneous gene excision by the Cre-loxP reaction was confirmed. Meanwhile, in iPS-234D-2, the extraneous gene excision was incomplete.

Hence, whichever of the wild type loxP sequences or mutant loxP sequences were used, it was shown that extraneous genes could be removed by Cre-loxP reaction, and that the morphology and function for iPS cells were retained even after the removal.

### Example 3

### Induction of IPS Cells from Mouse-Derived Fibroblasts (Exp. No. 283)

A mutant mouse having both a Nanog reporter and Fbx15 reporter was prepared by mating a Nanog reporter mouse (Okita K. et al., Nature 448, 313-317 (2007)) and an Fbx15 reporter mouse (Tokuzawa et al. Mol Cell Biol, Vol. 23, 2699-2708 (2003)). MEFs from this Fb/Ng reporter mouse were sown to a gelatin-coated 6-well culture plate at 1×10⁵ cells/well. In the same manner as Example 2, 3 different retrovirus vectors consisting of pMXs-Oct3/4-mloxP, pMXs-Sox2-mloxP and pMXs-Klf4-mloxP were introduced into the cells.

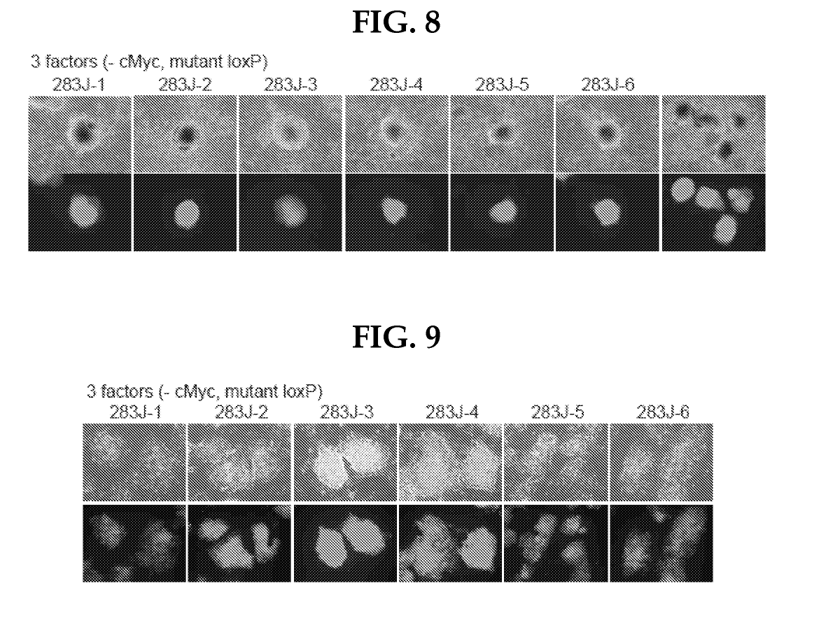

On day 25 of viral infection, selection with puromycin (1.5 μg/mL) was started. On day 33, colonies were picked up. Photographs of colonies as of the time of establishment are shown in FIG. 8. Photographs of colonies of the 2nd subculture are shown in FIG. 9. The colonies obtained exhibited a typical ES-cell-like morphology and tested positive for GFP, demonstrating the establishment of iPS cells.

Subsequently, the iPS cells of the 5th subculture were treated with Cre in the same manner as Example 2, and RT-PCR analysis was performed to confirm extraneous gene excision. The results are shown in FIG. 6 (right). For iPS-283J-3 and iPS-283J-4, extraneous gene excision by the Cre-loxP reaction was confirmed in all the clones examined.

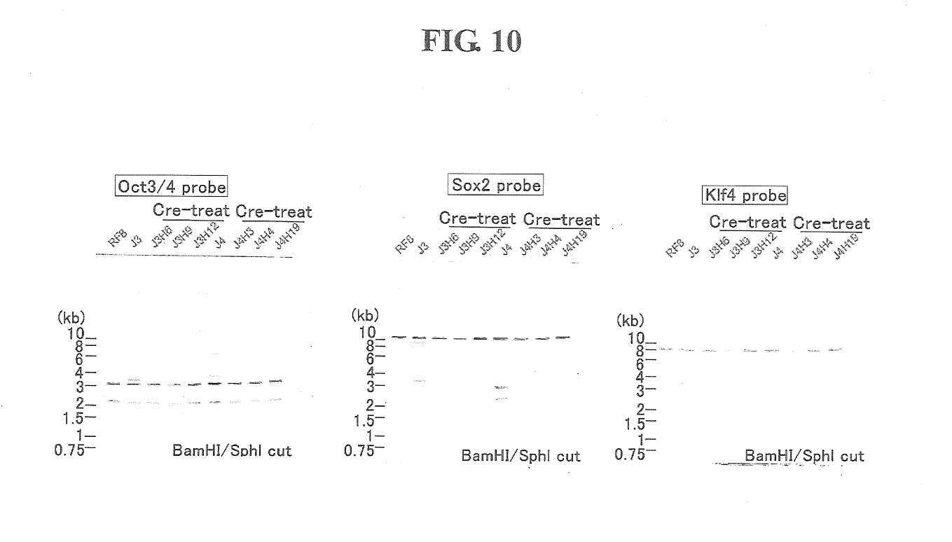

Furthermore, extraneous gene excision was confirmed by Southern blot analysis. The genomic DNA was extracted from each Cre-treated iPS clone and cleaved with BamHI/SphI, and this was followed by the Southern blot analysis. The results are shown in FIG. 10. Whichever of Oct3/4, Sox2 and Klf4 was used as a probe, the band from each transferred gene (extraneous gene) detected in iPS clones before Cre treatment (J3, J4) disappeared from each Cre-treated clone, confirming extraneous gene excision by the Cre-loxP reaction.

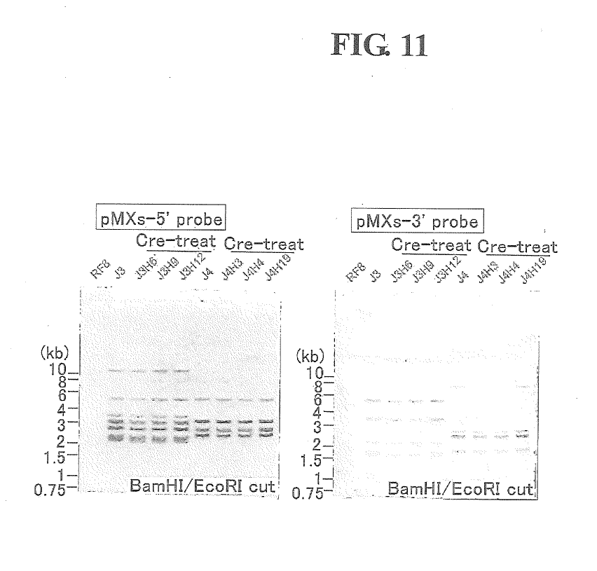

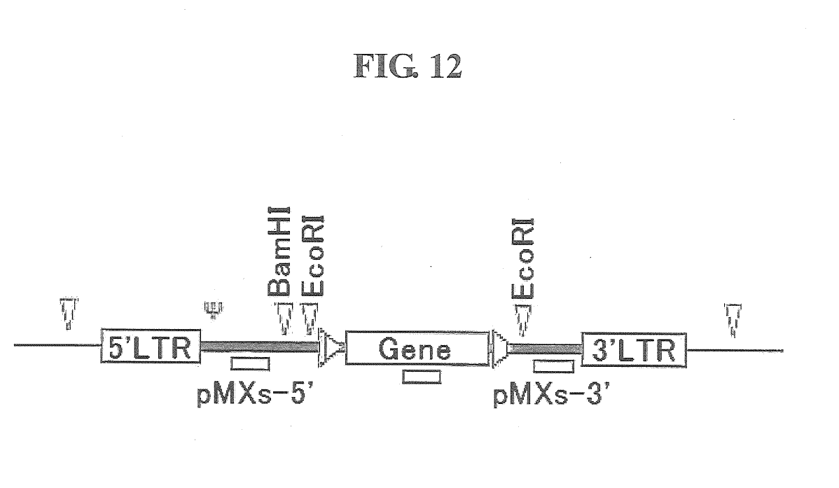

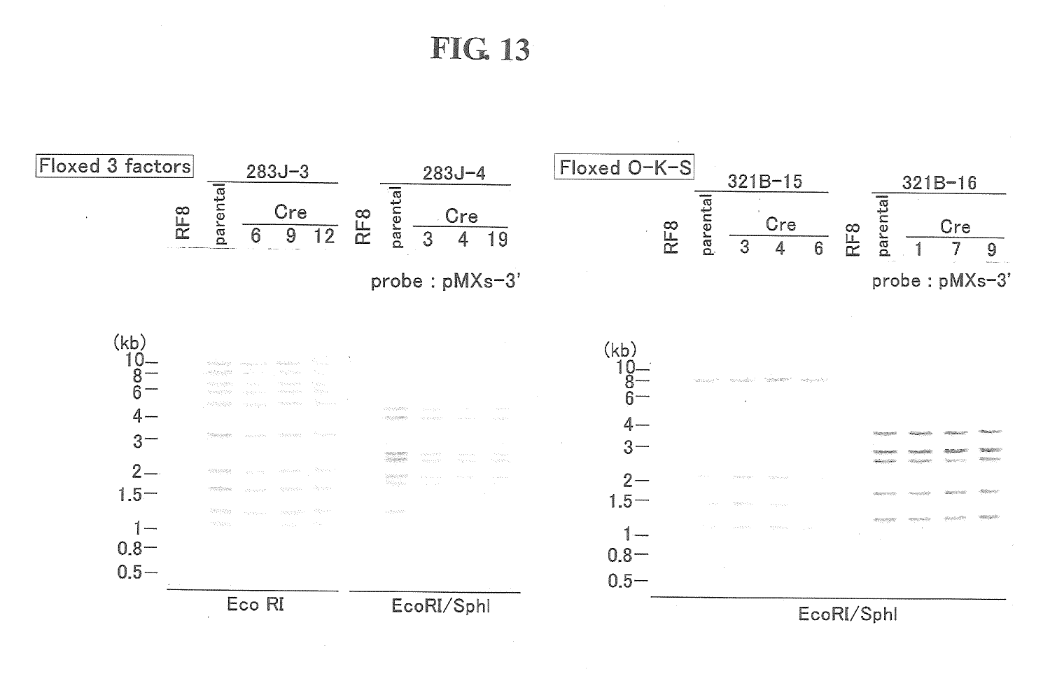

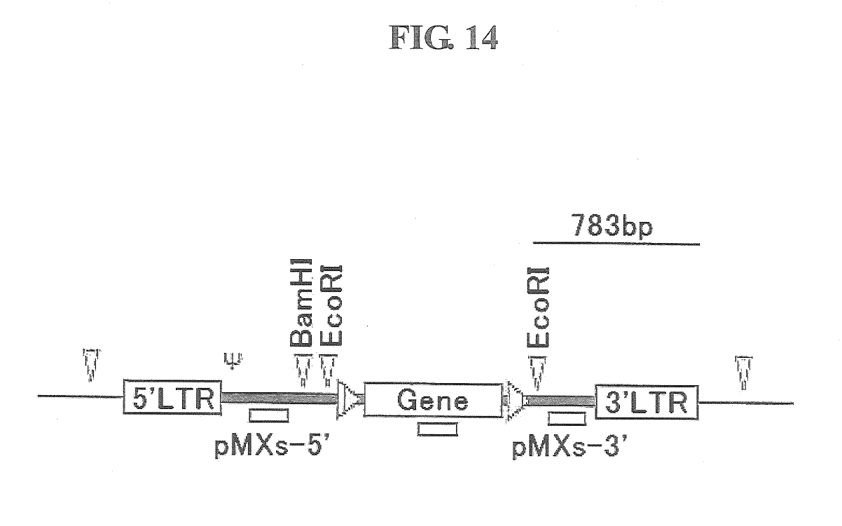

Subsequently, to confirm that the Cre-loxP reaction did not cause a deletion recombination reaction in the same chromosome, the genomic DNA of each Cre-treated iPS clone was cleaved with BamHI/EcoRI, and this was followed by Southern blot analysis. The results are shown in FIG. 11. A schematic diagram of the pMXs vector integrated in the genome showing the position of the probes is given in FIG. 12. The Cre-treated iPS clones exhibited the same band patterns as those from the iPS clones before Cre treatment (J3, J4), confirming that no recombination reaction occurred in the chromosome. The results of a Southern blot analysis of the same iPS clones, but cleaved with different restriction endonucleases (EcoRI for J3 series, EcoRI/SphI for J4 series), performed using the pMXs-3′ probe, are shown in FIG. 13 (left). A schematic diagram of the pMXs vector integrated in the genome showing the position of the probe is given in FIG. 14. Likewise, it was confirmed that no recombination reaction occurred in the chromosome.

### Example 4

### Induction of iPS Cells from Mouse-Derived Fibroblasts (Exp. No. 321)

MEFs from an Fb/Ng reporter mouse were sown to a gelatin-coated 6-well culture plate at 1×10⁵ cells/well. In the same manner as Example 2, the following retrovirus vectors were introduced into the cells.

(1) pMXs-OKS-mloxP
(2) pMXs-KOS-mloxP
(3) pMXs-OK-mloxP, pMXs-Sox2-mloxP
(4) pMXs-Oct3/4-mloxP, pMXs-Sox2-mloxP, pMXs-Klf4-mloxP

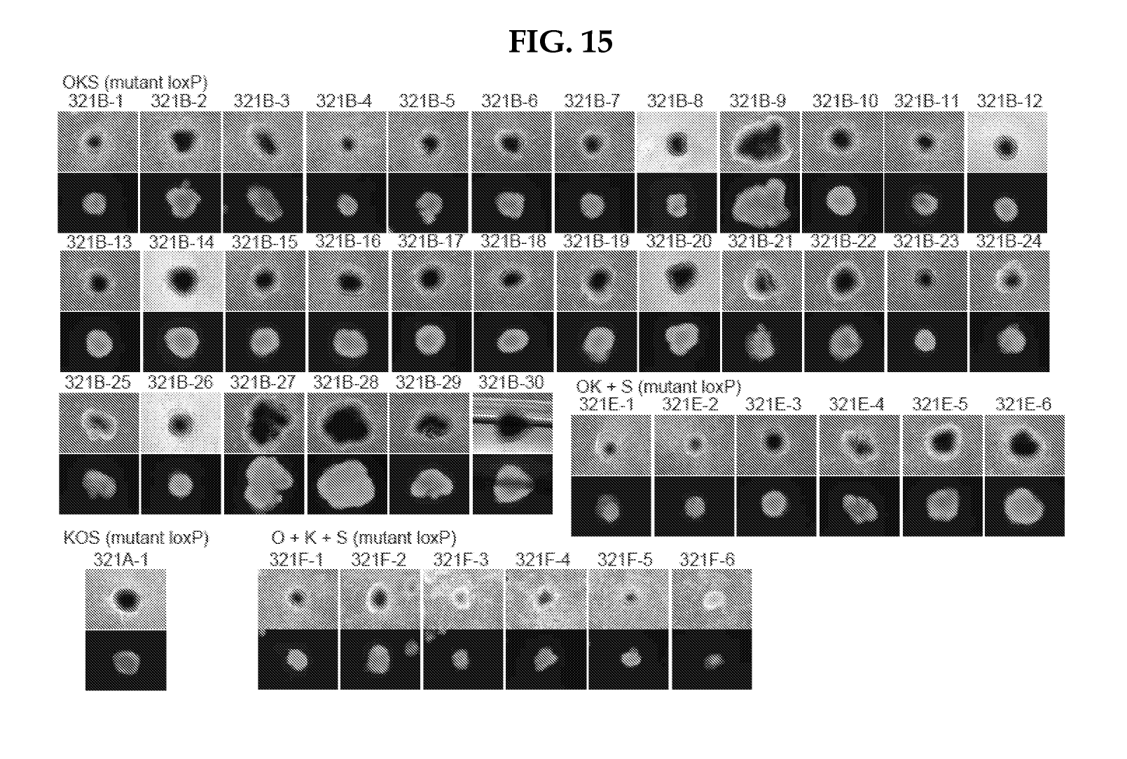

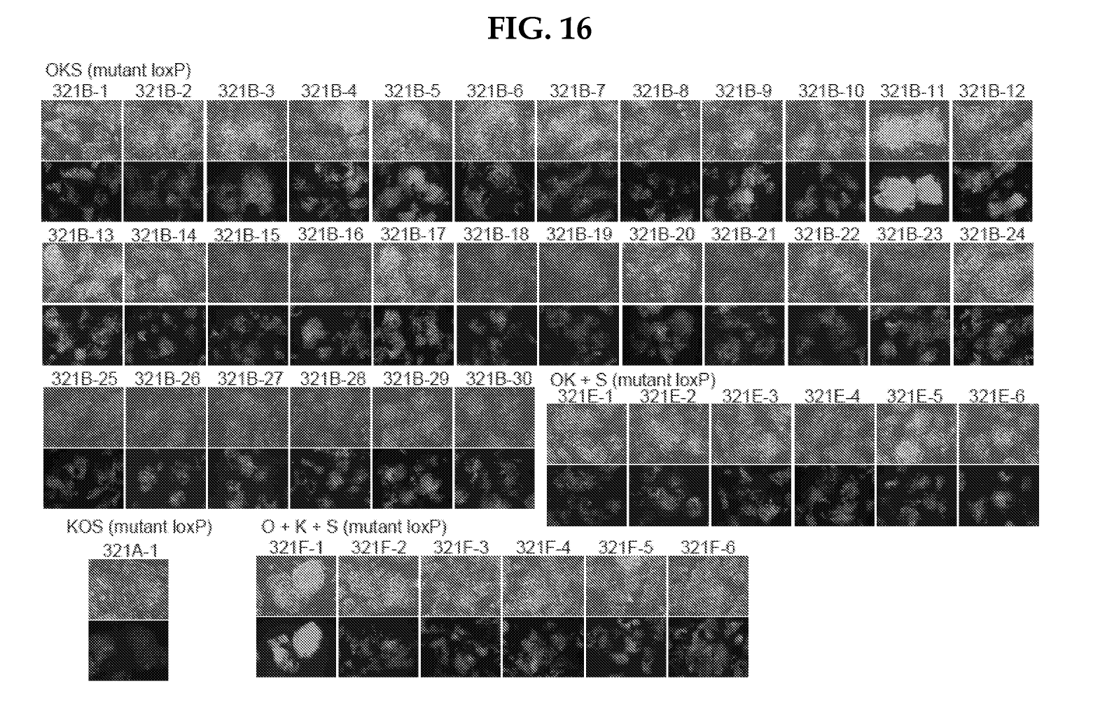

On day 21 of viral infection, selection with puromycin (1.5 μg/mL) was started. On day 28, colonies were picked up. Photographs of colonies as of the time of establishment are shown in FIG. 15. Photographs of colonies of the 2nd subculture are shown in FIG. 16. The colonies obtained exhibited a typical ES-cell-like morphology and tested positive for GFP, demonstrating the establishment of iPS cells.

Subsequently, 30 μg of pCAG-Cre-Hyg was electrically introduced into 1×10⁷ iPS cells of the 5th subculture (Bio-Rad Company: GenePulser Xcell). One-third of the cells were sown onto a 100 mm dish containing previously sown MSTO cells. On day 2 to day 4 after the transfection, the cells were treated with hygromycin, whereby Cre expressing cells were selected. On day 21, iPS colonies were picked up; after 1 subculture, genomic PCR analysis was performed to confirm extraneous gene excision by Cre.

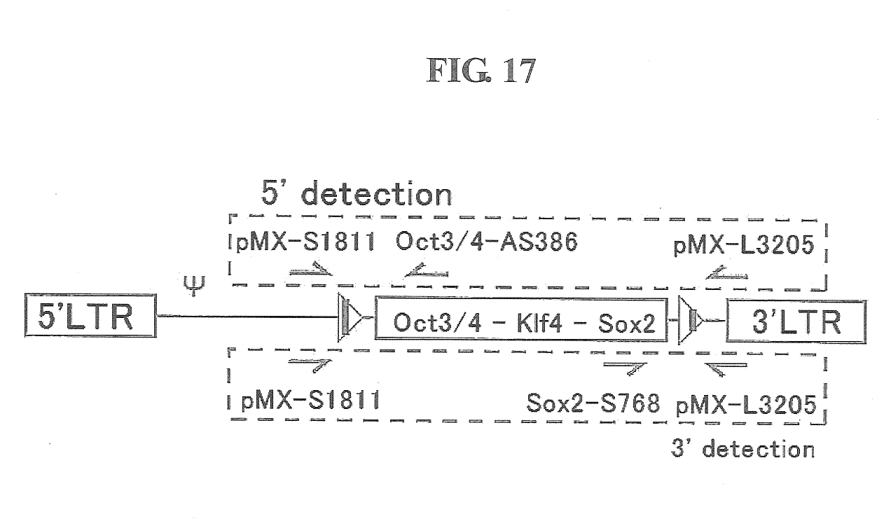

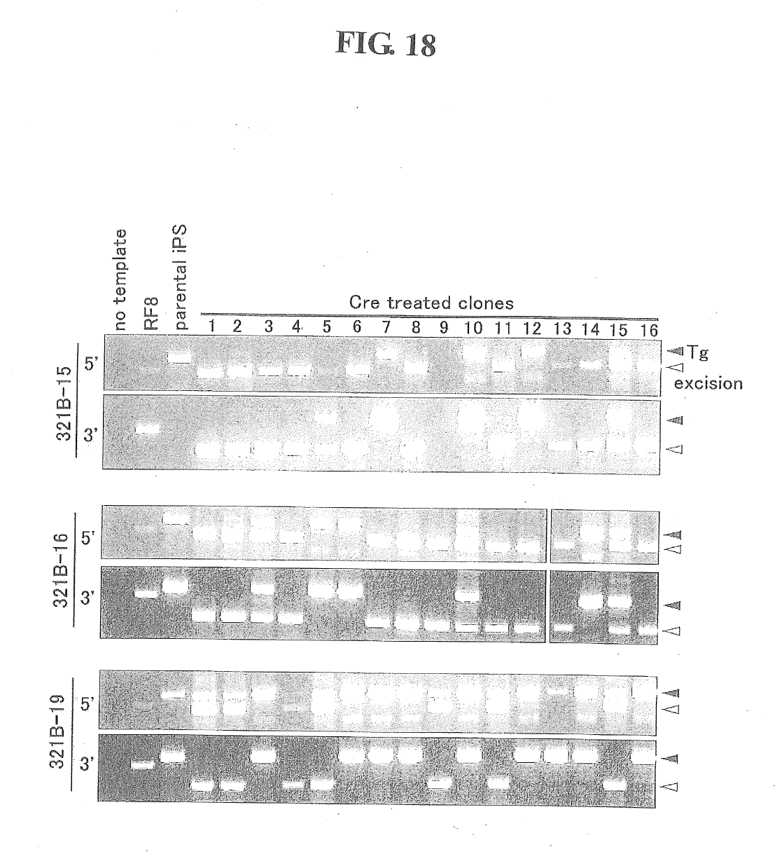

A schematic diagram of pMXs-OKS-mloxP showing the position of the primers used in the genomic PCR analysis is given in FIG. 17. The results of a genomic PCR analysis of Cre-treated iPS cells incorporating pMXs-OKS-mloxP are shown in FIG. 18. In Cre-treated 321B-15 and 321B-16, clones having the extraneous gene excised therefrom by the Cre-loxP reaction were detected (clones that produced a band only at the “excision” position in the figure).

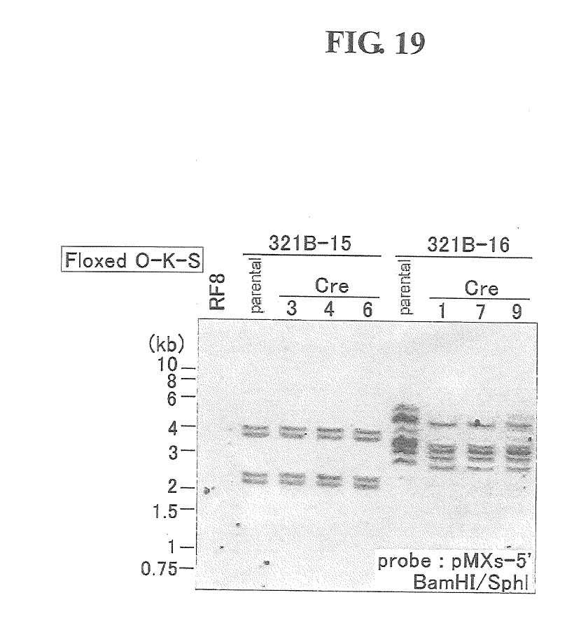

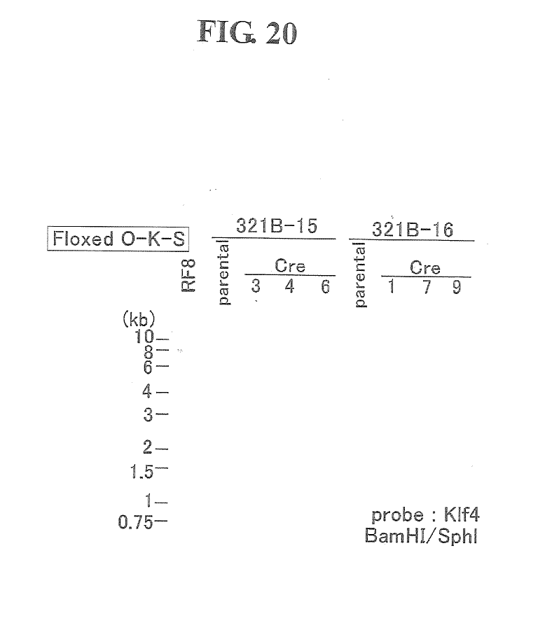

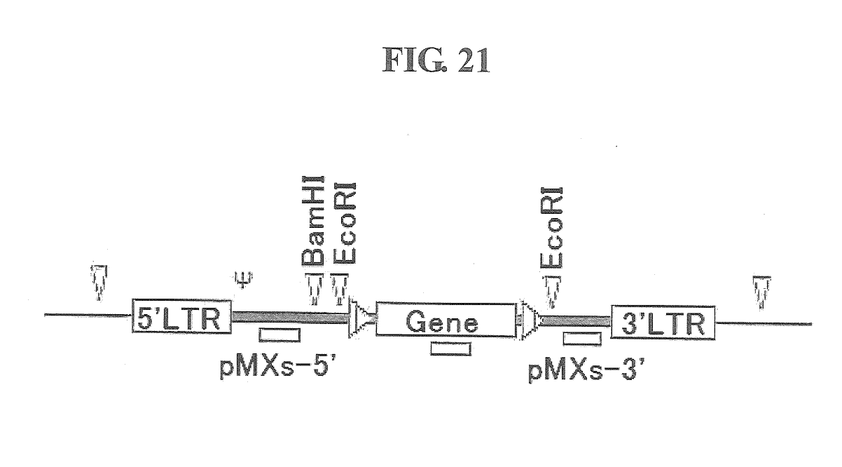

Subsequently, the genomic DNA was extracted from each of the iPS clones 321B-15 and 321B-16 wherein extraneous gene excision had been confirmed by genomic PCR, and this was followed by Southern blot analyses. The results of a Southern blot analysis using a 5′-side probe (pMXs-5′) by cleavage with BamHI/SphI are shown in FIG. 19. The results of a Southern blot analysis using a 3′-side probe (pMXs-3′) by cleavage with EcoRI/SphI are shown in FIG. 13 (right). The Cre-treated iPS clones exhibited the same band patterns as those from the iPS clones before the Cre treatment (parental), confirming that no deletion recombination reaction occurred in the chromosome. The results of a Southern blot analysis using Klf4 as a probe showed that the band from the extraneous gene detected in the iPS clone before the Cre treatment (parental) disappeared after the Cre treatment, confirming extraneous gene excision by the Cre-loxP reaction (FIG. 20). A schematic diagram of the pMXs vector integrated in the genome showing the positions of the respective probes is given in FIG. 21.

### Example 5

### Analysis of Chimeric Mice

iPS cells were established by transferring 3 different plasmids consisting of pMXs-Oct3/4-mloxP, pMXs-Sox2-mloxP, and pMXs-Klf4-mloxP, or 1 plasmid consisting of pMXs-OKS-mloxP, into MEF cells. Subsequently, the gene regions flanked by loxP sequences were removed from the iPS cells using Cre recombinase. These IPS cells (Examples 3 and 4) were micro-injected into early embryos from a wild type mouse, and chimeric mice were created. Long-term monitoring of 71 adult chimeras showed that they survived like normal mice for 1 year or more. This finding demonstrates that even the retention of intact LTRs on the genome after Cre cleavage of 3 genes consisting of Oct3/4, Klf4 and Sox2 does not lead to poor prognosis of the chimeric mice.

While the present invention has been described with emphasis on preferred embodiments, it is obvious to those skilled in the art that the preferred embodiments can be modified. The present invention intends that the present invention can be embodied by methods other than those described in detail in the present specification. Accordingly, the present invention encompasses all modifications encompassed in the gist and scope of the appended “CLAIMS.”

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the appended claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

This application is based on a U.S. provisional application Ser. No. 61/217,284, the contents of which are incorporated in full herein by this reference.

1. A method of preparing an iPS cell deprived of a reprogramming gene, comprising providing an iPS cell having an expression vector wherein a loxP sequence is placed on each of the 5′ and 3′ sides of the reprogramming gene or a vector component necessary for the replication of the reprogramming gene in the same orientation, and treating the iPS cell with Cre recombinase.

2. The method according to claim 1, wherein the iPS cell has on the chromosome thereof an expression vector wherein a loxP sequence is placed on each of the 5′ and 3′ sides of the reprogramming gene in the same orientation.

3. The method according to claim 2, wherein the vector is a retrovirus vector or lentivirus vector.

4. The method according to claim 3, wherein the loxP sequences are placed inward from the 5′ side LTR and 3′ side LTR, respectively.

5. The method according to claim 1, wherein the iPS cell has outside the chromosome thereof an expression vector wherein a loxP sequence is placed on each of the 5′ and 3′ sides of the vector component necessary for the replication of the reprogramming gene.

6. The method according to claim 5, wherein the vector is an episomal vector.

7. The method according to claim 6, wherein the episomal vector is derived from Epstein-barr virus.

8. The method according to claim 7, wherein the vector component necessary for the replication of the reprogramming gene is the replication origin oriP.

9. The method according to claim 7, wherein the vector component necessary for the replication of the reprogramming gene is a gene that encodes EBNA-1.

10. The method according to claim 6, wherein the episomal vector is derived from SV40.

11. The method according to claim 10, wherein the vector component necessary for the replication of the reprogramming gene is the replication origin Ori.

12. The method according to claim 10, wherein the vector component necessary for the replication of the reprogramming gene is a gene that encodes the SV40 large T antigen.

13. The method according to claim 1, wherein the loxP sequences are wild type loxP sequences (SEQ ID NO:1).

14. The method according to claim 1, wherein the loxP sequences are mutant loxP sequences.

15. The method according to claim 14, wherein the mutant loxP sequences are combinations of lox71 (SEQ ID NO:3) and lox66 (SEQ ID NO:4).

16. The method according to claim 1, wherein the reprogramming gene includes at least one gene selected from among Oct3/4, Sox2, Klf4, c-Myc, Nanog, Lin28 and the SV40 large T antigen.

17. The method according to claim 16, wherein the reprogramming gene is a polycistronically joined set of 2 or more genes selected from among Oct3/4, Sox2, Klf4, c-Myc, Nanog, Lin28 and the SV40 large T antigen.

18. The method according to claim 17, wherein the reprogramming gene is a polycistronically joined set of 3 different genes consisting of Oct3/4, Sox2 and Klf4 or 4 different genes consisting of Oct3/4, Sox2, Klf4 and c-Myc.

19. The method according to claim 17, wherein the genes are polycistronically joined via the 2A sequence of foot-and-mouth disease virus.

20. The method according to claim 1, wherein the Cre recombinase treatment is carried out by transferring a Cre recombinase expression vector into the iPS cell to allow the is enzyme to be produced transiently in the cell.

21. The method according to claim 20, wherein the Cre recombinase expression vector is a plasmid vector.

22. An iPS cell deprived of a reprogramming gene, wherein the cell is obtained by the method according to claim 1.

23. The iPS cell according to claim 22, wherein the cell has in the intact form the 5′- and 3′-side LTRs harbored by the viral vector.

24. A use of the iPS cell according to claim 22 in producing a somatic cell.

25. The iPS cell according to claim 22 as a cell source in producing a somatic cell.
